(19) United States
(12) Patent Application Publication
(10) Pub. No.: US 2005/0175544 A1
(43) Pub. Date: Aug. 11, 2005

(54) Albuterol inhalation solution, system, kit and method for relieving symptoms of pediatric asthma

(75) Inventors: Imtiaz Chaudry, American Canyon, CA (US); Partha Banerjee, Plainsboro, NJ (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Dey LP

(21) Appl. No.: 11/013,491

(22) Filed: Dec. 15, 2004

Related U.S. Application Data
(63) Continuation-in-part of application No. 10/274,467, filed on Oct. 18, 2002, now abandoned.
Application No. 10/274,467 is a continuation-in-part of application No. 10/034,829, filed on Dec. 28, 2001, now Pat. No. 6,702,997.
(60) Provisional application No. 60/348,203, filed on Oct. 26, 2001.

(51) Int. Cl. A61L 9/04; A61K 31/137

(52) U.S. Cl. 424/45; 514/649

(57) ABSTRACT

The present invention relates to an albuterol inhalation solution, system, kit and method for relieving bronchospasm in children suffering from asthma. In one alternative embodiment, the solution of the present invention is a sterile, premixed, premeasured single unit dose of albuterol for asthmatic patients 2 to 12 years of age. The present solution may be free of anti-microbial preservatives, such as benzalkonium chloride. In another alternative embodiment, the solution of the present invention comprises about 0.75 mg or about 1.5 mg albuterol sulfate.

### I. CROSS REFERENCE TO RELATED APPLICATIONS

This application is a continuation-in-part of U.S. application Ser. No. 10/274,467, filed Oct. 18, 2002, which is a continuation-in-part of U.S. application Ser. No. 10/034,829, filed Dec. 28, 2001 (now U.S. Pat. No. 6,702,997) which claims priority under 35 U.S.C. §119 (e) from U.S. Provisional Application Ser. No. 60/348,203 filed Oct. 26, 2001. The entire disclosure of the above-cited prior applications are incorporated herein by reference in their entirety.

### II. FIELD OF THE INVENTION

The present invention relates to an albuterol inhalation solution, system, kit and method for relieving symptoms associated with asthma in children.

### III. BACKGROUND OF INVENTION

Asthma is a pulmonary disease marked by (1) labored breathing; (2) wheezing; and (3) coughing. Asthma is characterized by: (1) airway inflammation; (2) airway hyperresponsiveness; and (3) airway obstruction (or airway narrowing) that is partially or completely reversible, either spontaneously or with treatment. Common symptoms of asthma include wheezing, shortness of breath, tightness in the chest and a persistent cough. The severity of the symptoms vary widely from patient to patient, and even from one episode (attack) to the next.

A key condition of asthma is chronic inflammation of the linings of the lungs. This inflammation is associated with an increase in airway sensitivity (hyperresponsiveness) to stimuli such as allergens, irritants, cold air and viruses. When exposed to these triggers, the linings undergo an allergic reaction, causing spasms that constrict the airways. This bronchoconstriction, in combination with edema and the release of thickened secretions, reduces movement of air through the lungs, resulting in the symptoms commonly associated with asthma.

Asthma is the most common chronic lung disease in children. Asthma prevalence in children has reportedly increased in the United States by 160%. Asthma hospitalization rates are also higher in young children due, in part, to difficulties in using currently available drug delivery devices and failure to use optimal doses of asthma therapies.

Despite progress in emergency and critical care medicine, the pediatric mortality rate from asthma ranges from 0.2 to 0.4 per 100,00 population, depending on age. Pediatric asthma ranks as the 7^{th} leading cause of death among children ages 10 to 14 years. Approximately 0.05% of known children with asthma die annually from the disease.

Short-acting inhaled beta-agonists, such as albuterol, are the first choice treatment for relieving symptoms of acute asthma in children. Albuterol is currently available as a 2.5 mg unit dose (0.083%) inhalation solution for use in nebulizers. Although this dose has been approved for use by adults, the FDA has recently expanded labeling guidelines to include this amount of albuterol for use by pediatric asthmatic patients as young as 2 years old. However, when administered on a regular basis to a child, the 2.5 mg formulation may provide more albuterol than needed, thereby increasing the risk of adverse drug effects.

In the recently revised guidelines for asthma treatment, the National Institutes of Health recommended that pediatric patients use the lowest beta-agonist doses needed to control symptoms. However, using lower doses of albuterol in patients under age 12 to reduce the risk of side effects necessitates dilution of currently available asthma medications. This poses several problems because parents, care givers, teachers and others typically do not have adequate experience diluting these medications, resulting in contamination or inappropriate dosing, among other problems.

Also, antimicrobial preservatives, such as benzalkonium chloride (BAC), are often present in inhalation solutions used to treat asthma and chronic obstructive pulmonary disease (COPD). The presence of BAC in these solutions generally does not affect the short-term (single dose) bronchodilator response. However, case reports suggest that repeated use of asthma treatments with BAC may result in paradoxic bronchoconstriction. When inhaled by asthmatic subjects, BAC may also cause dose-dependent bronchoconstriction. Despite these side effects, many commercially available albuterol inhalation solutions contain BAC.

There is, therefore, a need for an improved albuterol inhalation solution, system, kit and method for relieving symptoms associated with pediatric asthma.

### IV. SUMMARY OF THE INVENTION

One object of the present invention is to provide an albuterol inhalation solution for the relief of bronchospasm in children with asthma. Another object of the present invention is to provide a prepackaged, sterile, premixed, premeasured, reduced-dosage albuterol inhalation solution for the relief of bronchospasm in patients 2 to 12 years of age with asthma.

It is yet another object of the present invention to provide an antimicrobial preservative-free albuterol inhalation solution to relieve bronchospasm in a pediatric patient with asthma.

A further object of the present invention is to provide a method of administering an albuterol inhalation formulation for relief of bronchospasm associated with pediatric asthma.

An additional object of the present invention is to provide a kit or system for relief of bronchospasm in a pediatric patient with asthma.

A further object of the present invention is to provide a process for making an inhalation solution for relief of bronchospasm in a pediatric patient with asthma.

Another object of the invention includes a device for use in relieving bronchospasm in a pediatric patient with asthma.

Other objects, features and advantages of the present invention will be apparent to those of ordinary skill in the art in view of the following detailed description of the invention and accompanying drawings.

### V. BRIEF DESCRIPTION OF THE DRAWINGS

FIGS. 1-4 depict a non-limiting example of administering the inhalation solution of the present invention by a nebulizer.

FIG. 5 depicts a non-limiting example of a unified prepackaged kit or system of the present invention.

FIG. 6 depicts a non-limiting example of one or more pre-filled containers comprising the inhalation system of the present invention.

FIG. 7 depicts a non-limiting example of a label utilized in the present invention.

### VI. DETAILED DESCRIPTION OF THE INVENTION

Albuterol

The present invention relies on the bronchodilation effects of albuterol to provide relief from symptoms associated with pediatric asthma. As used herein, the term “albuterol” includes, but is not limited to, any form of albuterol which is capable of producing a desired bronchodilation effect in pediatric patients, including, but not limited to, all tautomeric forms, enantomeric forms, stereoisomers, anhydrides, acid addition salts, base salts, solvates, analogues and derivatives of albuterol.

In the present invention, acceptable salts of albuterol may include, but are not limited to, hydrochloride, sulfate, maleate, tartrate, citrate and the like. These and other acceptable salts are described in U.S. Pat. No. 3,644,353, which is incorporated herein by reference in its entirety.

In the present invention, the preferred salt of albuterol is sulfate. In an alternative embodiment, the inhalation solution of the present invention comprises the sulfate salt of racemic albuterol. Albuterol sulfate is a relatively selective beta-2-adrenergic bronchodilator with an empirical formula of C₁₃H₂₁NO₃. The chemical name for albuterol sulfate is α¹-[(tert-butylamino)methyl]-4-hydroxy-m-xylene-α,α′-diol sulfate (2:1)(salt), and its established chemical structure is as follows:

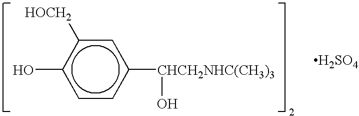

In the present invention, the albuterol may be provided in a variety of pharmaceutically acceptable vehicles, including, but not limited to, water or other aqueous solutions comprising a pharmaceutically acceptable amount of an osmotic agent.

In one alternative embodiment, the inhalation solution of the present invention comprises a therapeutically effective pediatric amount of albuterol. As used herein the phrase “therapeutically effective pediatric amount of albuterol” means a safe and tolerable amount of albuterol for pediatric patients, as based on industry and/or regulatory standards. Such amount being sufficient to effectively induce bronchodilation and/or provide relief of bronchospasm in children.

In the inhalation solution of the present invention, a therapeutically effective pediatric amount of albuterol may include about 0.63 mg or about 1.25 mg albuterol. Here, the potency of the albuterol is equivalent to about 0.75 mg and about 1.50 mg of albuterol sulfate, respectively. In an alternative embodiment, a therapeutically effective pediatric amount of albuterol may include from about 0.63 mg to about 1.25 mg of albuterol. In another alternative embodiment, such pediatric amount comprises no more than about 1.25 mg of albuterol, or it comprises 1.25 mg of albuterol or less.

In another alternative embodiment of the present invention, a therapeutically effective pediatric amount of albuterol may include from about 0.08 mg to about 1.90 mg albuterol, including the following intermediate amounts of albuterol: about 0.08 mg to about 0.20 mg; about 0.21 mg to about 0.50 mg; about 0.51 mg to about 0.60 mg; about 0.61 mg to about 0.70 mg; about 0.71 mg to about 0.80 mg; about 0.81 mg to about 0.90 mg; about 0.91 mg to about 1.0 mg; about 1.01 mg to about 1.05 mg; about 1.06 mg to about 1.10 mg; about 1.11 mg to about 1.15 mg; about 1.16 mg to about 1.20 mg; about 1.21 mg to about 1.25 mg; about 1.26 mg to about 1.30 mg; about 1.31 mg to about 1.35 mg; about 1.36 mg to about 1.40 mg; about 1.41 mg to about 1.45 mg; about 1.46 mg to about 1.50 mg; about 1.51 mg to about 1.55 mg; about 1.56 mg to about 1.60 mg; about 1.61 mg to about 1.65 mg; about 1.66 mg to about 1.70 mg; about 1.71 mg to about 1.75 mg; about 1.76 mg to about 1.80 mg; about 1.81 mg to about 1.85 mg; about 1.86 mg to about 1.90 mg.

In another alternative embodiment of the present invention, a therapeutically effective pediatric amount of albuterol may include from about 0.1 mg to about 2.5 mg albuterol sulfate, including the following intermediate amounts of albuterol sulfate: about 0.1 mg to about 0.2 mg; about 0.3 mg to about 0.4 mg; about 0.5 mg to about 0.6 mg; about 0.7 mg to about 0.8 mg; about 0.9 mg to about 1.00 mg; about 1.01 mg to about 1.20 mg; about 1.21 mg to about 1.40 mg; about 1.41 mg to about 1.60 mg; about 1.61 mg to about 1.80 mg; about 1.81 mg to about 2.00 mg; about 2.01 mg to about 2.20 mg; about 2.21 mg to about 2.40 mg; about 2.41 mg to about 2.50 mg.

In another alternative embodiment of the present invention, a therapeutically effective pediatric amount of albuterol may include from about 0.002% to about 0.075% by weight albuterol, including the following intermediate amounts of albuterol: about 0.002 wt % to about 0.010 wt %; about 0.011 wt % to about 0.020 wt %; about 0.021 wt % to about 0.030 wt %; about 0.031 wt % to about 0.040 wt %; about 0.041 wt % to about 0.050 wt %; about 0.051 wt % to about 0.060 wt %; about 0.061 wt % to about 0.070 wt %; about 0.071 wt % to about 0.075 wt %.

In another alternative embodiment of the present invention, a therapeutically effective pediatric amount of albuterol may include from about 0.003% to about 0.1% by weight albuterol sulfate in solution, including the following intermediate amounts of albuterol sulfate: about 0.003 wt % to about 0.010 wt %; about 0.011 wt % to about 0.020 wt %; about 0.021 wt % to about 0.030 wt %; about 0.031 wt % to about 0.040 wt %; about 0.041 wt % to about 0.050 wt %; about 0.051 wt % to about 0.060 wt %; about 0.061 wt % to about 0.070 wt %; about 0.071 wt % to about 0.080 wt %; about 0.081 wt % to about 0.090 wt %; about 0.091 wt % to about 0.10 wt %.

Most pharmaceutical inhalation solutions contain an antimicrobial preservative such as BAC or EDTA. One problem with BAC-containing solutions is that the BAC may cause paradoxic bronchoconstriction if the solution is administered repeatedly over short intervals.

Another problem is that, when inhaled by asthmatic patients, the BAC can cause dose-dependent bronchoconstriction. The inhalation solution of the present invention may be provided without BAC, thereby making it more suitable for pediatric patents, especially in an emergency situation where the inhalation solution is administered repeatedly over a short period of time. Also, administering a BAC-free inhalation solution to a pediatric patient reduces the concomitant liability of adverse effects associated with BAC. It also reduces the toxicity and other side effects associated with BAC.

The inhalation solution of the present invention may also be provided in sterile, unit dose treatments, thus eliminating the need to include BAC in the solution. Moreover, as shown in Table 1, in its sterile form the formulation of the present invention (which comprises a therapeutically effective pediatric amount of albuterol) provides a stable pediatric inhalation solution such that the formulation can be stored (e.g., on a shelf) for long periods of time.

TABLE 1
Stability Data

| | | 0.021 wt % Albuterol | | | 0.042 wt % Albuterol | | |
|---|---|---|---|---|---|---|---|
| | | | | Osmolality | | | Osmolality |
| | | Assay* | pH | (mOsm/kg) | Assay* | pH | (mOsm/kg) |
| Time zero | | 98 | 3.5 | 289 | 100 | 3.5 | 291 |
| 25° C./35% RH | 12 months | 99 | 3.5 | 289 | 100 | 3.5 | 291 |
| | 24 months | 101 | 3.5 | 294 | 100 | 3.5 | 292 |
| 40° C./15% RH | 3 months | 99 | 3.6 | 290 | 100 | 3.6 | 291 |
| | 6.5 months | 96 | 3.5 | 290 | 99 | 3.5 | 292 |

*as percent of label claim (0.021 wt % and 0.042 wt % albuterol, respectively).

Another benefit of a sterile inhalation solution is that it reduces the possibility of introducing contaminants into the patient when administered, thereby reducing the chance of an opportunistic infection in the patient.

As stated, the compositions provided herein are stable. For example, the compositions provided herein are stored between about 15° C. and about 30° C., and remain stable for a relatively long period of time. In one embodiment, the compositions are stored at 25° C.

In another embodiment, the stability of the compositions provided herein may contain greater than 80%, 85%, 90% or 95% of the initial amount of active ingredient, i.e., Albuterol, at a given temperature for a long period of time. Thus, for example, a composition that is stable for 30 days at 25° C. would have greater than 80%, 85%, 90% or 95% of the initial amount of active ingredient present in the composition at 30 days following storage at 25° C.

In another embodiment, the compositions herein are stable during long term storage, in that the compositions are suitable for administration to a subject in need thereof when the compositions have been stored for a length of time (i.e., shelf-life) for a period greater than 1, 2 or 3 years at 25° C. In other embodiments herein, using Arrhenius Kinetics, >80% or >85% or >90% or >95% estimated bronchodilating agent remains after such storage, for example.

Other indications of the stability of the present compositions can be shown in terms of by-products or degradation products present over time, as shown in Table 2 below.

TABLE 2

| | | Range at | |
|---|---|---|---|
| | | 6 to 18 | Range |
| Degradation products/related | | months | in drug |
| compounds as % of albuterol | | at 25° C. | substance |
| 1 | 5-2-((1,1-Dimethylethyl)amino- | ND-0.012% | |
| | 1-hydroxyethyl)-2- | w/w | |
| | hydroxybenzaldehyde | | |
| 2 | Bis-(2-hydroxy0-5-(2- | | 0.09-0.174% |
| | tertbutylamino-1-hydroxy- | | w/w |
| | ethyl)phenylmethyl ether | | |
| 3 | 2-tert-butylamino-1-(4- | | 0.01-0.12% |
| | hydroxy-3-methoxymethyl- | | w/w |
| | phenyl)-ethanol | | |
| 4 | Tert-butylamino-3-chloro- | | ND-0.0002% |
| | 4-hydroxy-5-hydroxymethyl- | | w/w |
| | acetophenone | | |
| 5 | Tert-butylamino-4-hydroxy- | | ND-0.002% |
| | 5-hydroxymethylacetophenone | | w/w |
| 6 | 1-(4-hydroxy-3-methylphenyl)- | | 0.0009-0.036% |
| | 2-(tert-butylamino)ethanol | | w/w |
| 7 | 1-(5-chloro-4-hydroxy-3- | | ND |
| | hydroxymethylphenyl)-2- | | |
| | (tert-butylamino)ethanol | | |
| 8 | Any other unknown | ND-0.06% | |
| | | by peak area | |
| 9 | Total | 0.1-0.38% | |

ND = none detected

In one embodiment, the compositions produced herein are at least substantially clear, based on color measurement tests set forth by the America Public Health Association (“APHA”). For example, the APHA color results for compositions herein at upto 24 months at 25° C. may range from less than 10 units, or preferably 0 to 5 units, most preferably 0 units as based on APHA standards.

In one embodiment, the process of the present invention provides compositions having an albuterol content of about 0.021% or 0.042% per vial. In another alternative embodiment, the process of the present invention provides compositions having an albuterol content of about 0.0197% to about 0.0218% w/v, about 0.0201% to about 0.0214% w/v, about 0.0394% to about 0.0436% w/v and about 0.0403% to about 0.0428% w/v per vial. In yet another alternative embodiment, the process of the present invention provides an average fill volume of about 2.80 ml to about 3.30 ml into each vial.

In another alternative embodiment, the process of the present invention provides compositions that may contain minimal amounts of contaminants including, but not limited to the following:

TABLE 3

| | |
|---|---|
| 1. Volatiles | |
| acetone | none detected to about NMT 0.2 mcg/ml |
| ethyl acetate | none detected to about NMT 0.3 mcg/ml |
| n-heptane | none detected to about NMT 0.1 mcg/ml or less |
| n-propyl acetate | none detected to about NMT 0.3 mcg/ml |
| toluene | none detected to about NMT 0.1 mcg/ml |
| 2-butanone | none detected to about NMT 0.3 mcg/ml |
| unknowns | |
| 2. Leachables | |
| Irganox 129 | none detect to about NMT 0.02 mcg/ml |
| Extractable 1 | none detected (signal/noise NMT 3) |
| Extractable 2 | none detected (signal/noise NMT 3) |
| unknowns | none detected (signal/noise NMT 3) |

In another alternative embodiment, such compositions may also contain minimal amounts of particulate matter, including, but not limited to the following: about NMT 1000 to 5000 particles, preferably about NMT 3800 particles/vial >2mm; about NMT 10 to about 100 particles, preferably about 80 particles/vial>10 mcm; or about NMT 1 to about 5 particles, preferably about NMT 3 particles/vial>25 mcm.

Adherence to asthma medication therapy and prevention of asthma medication error are considerable problems. These problems can be significantly reduced by providing asthmatic patients a prepackaged, premixed, premeasured amount of albuterol. Providing albuterol in this fashion makes asthma therapy simple because it increases convenience and eliminates confusion in preparing appropriate dosages. These advantages are especially significant in the treatment of pediatric asthma, where treatments often come in multiple dosage units and must be diluted to specific concentrations suitable for treating a pediatric patient. This poses several problems. For instance, asthma treatments requiring administration of a single dose unit from multiple dosage units sometimes lack proper mixing or diluting instructions, or the instructions for preparing and using the asthma treatment may be hard to follow or can be easily lost. Of even greater importance is haphazard diluting or mixing of asthma medications, which can result in administering the wrong dosage. This could be especially harmful for pediatric patients, who often are less tolerant to higher dosages of albuterol. Incorrect mixing can also result in treatment failure such that additional medical attention is required, thereby increasing the time, expense, and personnel costs associated with therapy.

The present invention overcomes the aforementioned problems by providing therapeutically effective pediatric amounts of albuterol in prepackaged, premixed, premeasured and/or unit dose amounts. In one embodiment, the present invention comprises one or more prefilled containers. The one or more containers each comprising a single unit dose of an aqueous solution comprising a therapeutically effective pediatric amount of albuterol for the relief of bronchospasm associated with pediatric asthma. Providing the inhalation solution in such a manner eliminates the need to dilute or mix asthma medications to obtain proper dosages for treatment. Also, no special pharmacy compounding is required, and the chance of medication errors are reduced. Further, there is a lower risk of cross-contamination, and less waste of medication when providing an inhalation solution in a premixed, ready to use form.

Other features of the present invention include improved user compliance and quality of life as compared to conventional treatments for relieving bronchospasm in children. While the level of compliance of any asthma treatment depends in part on the motivation and skill of the individual dispensing the treatment, compliance nevertheless may be improved by controllable factors such as the ease with which the treatment may be administered, as well as the desirability of receiving the treatment.

The present invention provides a convenient, fast and reliable treatment for relieving bronchospasm in children, and clearly represents an improvement over traditional asthma treatments. Also, the present invention is designed to facilitate user compliance by providing one or more dispensing containers comprising a premixed, premeasured inhalation solution comprising a single unit dose of a therapeutically effective pediatric amount of albuterol for the relief of bronchospasm in children. Said containers may be utilized in a method of relieving such bronchospasm, or the containers may be incorporated in a system and/or kit for treating the same.

In one alternative embodiment, the formulation of the present invention is a sterile, premixed, premeasured, BAC-free inhalation solution comprising a single unit dose of a therapeutically effective pediatric amount of albuterol in a single container. Each unit dose container comprises either 0.75 mg/3 ml of albuterol sulfate (equivalent to 0.63 mg of albuterol) or 1.50 mg/3 ml of albuterol sulfate (equivalent to 1.25 mg of albuterol) in a sterile, aqueous solution. Sodium chloride may be added to adjust the isotonicity of the solution and sulfuric acid may be added to adjust pH of the solution to about 3.5. The inhalation solution of the present invention may or may not include a chelating agent, such as EDTA.

In another alternative embodiment, the inhalation solution of the present invention may be supplied as a 3 ml, sterile, BAC-free, nebulizer solution comprising from about 0.75 mg/3 ml to about 1.50 mg/3 ml of albuterol sulfate (equivalent to about 0.63 mg to about 1.25 mg of albuterol, respectively). The nebulizer solution is contained in a unit-dose, low-density polyethylene (LDPE) container. Each unit-dose container may be disposed in a foil pouch, and each foil pouch may contain 5 or more unit-dose containers. Each foil pouch containing the unit dose container may be disposed in a shelf carton.

The present invention provides an albuterol inhalation solution for relieving bronchospasm in a pediatric patient with asthma, including, but not limited to, allergic (extrinsic) asthma, non-allergic (intrinsic) asthma, occupational asthma and aspirin sensitive asthma. The present invention also provides an albuterol inhalation solution for relieving bronchospasm associated with different classes of pediatric asthma including, but not limited to, severe persistent asthma, moderate persistent asthma, mild persistent asthma and mild intermittent asthma. Some characteristics associated with the different classes of asthma are shown in Table 4. The information in this table is presented for illustrative purposes only. It is not intended to limit the scope of the invention.

In another embodiment of the present invention, the inhalation solution may have a pH of about 2.0 to about 8.0. In another embodiment of the claimed invention, the solution may have a pH of about 3.0 to about 4.0, preferably a pH of about 3.5. The pH may be adjusted with 1N hydrochloric acid or 1N sulfuric acid. The inhalation solution of the present invention may also contain sodium citrate at a concentration of about 0.1 to 0.5% (w/w), preferably about 0.2% (w/w/) to control pH or may further contain a buffer. General and biological buffers in the pH range of about 2.0-8.0 include but are not limited to the following: acetate, barbital, borate, Britton-Robinson, cacodylate, citrate, collidine, formate, maleate, Mcllvaine, phosphate, Prideaux-Ward, succinate, citrate-phosphate-borate (Teorell-Stanhagen), veronal acetate, MES, BIS-TRIS, ADA, ACES, PIPES, MOPSO, BIS-TRIS PROPANE, BES, MOPS, TES, HEPES, DIPSO, MOBS, TAPSO, TRIZMA, HEPPSO, POPSO, TEA, EPPS, TRICINE, GLY-GLY, BICINE, HEPBS, TAPS, and AMPD buffers.

In another embodiment of the present invention, the osmolality of the inhalation solution may be adjusted from about 150 to about 550 mOsm/kg. In other embodiments of the present invention, the osmolality of the solution may be from about 275 to about 325 mOsm/kg. In yet another embodiment, the composition may have an osmolality of about 290 mOsm/kg. Tonicity adjusting agents include but are not limited to the following excipients: ammonium carbonate, ammonium chloride, ammonium lactate, ammonium nitrate, ammonium phosphate, amonium sulfate, ascorbic acid, bismuth sodium tartrate, boric acid, calcium chloride, calcium disodium edetate, calcium gluconate, calcium lactate, citric acid, dextrose, diethanolamine, dimethyl sulfoxide, edetate disodium, edetate trisodium monohydrate, fluorescein sodium, fructose, galactose, glycerin, lactic acid, lactose, magnesium chloride, magnesium sulfate, manitol, polyethyne glycol, potassium acetate, potassium chlorate, potassium chloride, potassium iodide, potassium nitrate, potassium phosphate, potassium sulfate, propylene glycol, silver nitrate, sodium acetate, sodium bicarbonate, sodium biphosphate, sodium bisulfite, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine, and zinc sulfate.

The inhalation solutions provided herein may also include other excipients and additives. The excipients and additives include, but are not limited to, surfactants, stabilizers, complexing agents, antioxidants, viscosity-enhancing agents or preservatives which prolong the duration of use of the pharmaceutical formulation or modify the nebulization property of the composition. Complexing agents include but are not limited to, ethylenediaminetetraacetic acid (EDTA) or a salt thereof, such as the disodiumsalt, citric acid, nitrilotriacetic acid and the salts thereof. Preservatives may include, but are not limited to, benzalkonium chloride, benzoic acid and sodium benzoate. Antioxidants include, but are not limited to, vitamins, provitamins, ascorbic acid, vitamin E or salts or esters thereof, sodium sulfite or sodium metabisulfite.

In another alternative embodiment, the inhalation solution of the present invention may be administered by nebulizer. Such nebulizer including, but not limited to, a jet nebulizer, ultrasonic nebulizer and breath actuated nebulizer. Preferably, the nebulizer is a jet nebulizer connected to an air compressor with adequate air flow. The nebulizer being equipped with a mouthpiece or suitable face mask. Specifically, a Pari-LC-Plus™ nebulizer (with face mask or mouthpiece) connected to a PRONEB™ compressor may be used to deliver the inhalation solution of the present invention to a patient. In an embodiment, the inhalation solution may be administered by nebulizers manufactured, designed or sold by Omron, such as the Omron Micro Air™ Ultrasonic Nebulizer. Other nebulizers may also include those manufactured, designed, or sold by Aerogen.

Drugs administered by nebulization play a major role in the treatment of pediatric asthma. It has been shown that some patients have difficulty inhaling sufficient amounts of the prescribed medication from a nebulizer and this may be a reason for treatment failure. However, one of the drawbacks of nebulization therapy is the number of times it must be performed each day, and the amount of time each treatment takes. For example, an individual may be required to receive 4 doses of inhalation solution per day by nebulization. In some instances, each nebulizer treatment takes about 15 minutes, or more to deliver a 2.5 ml fill volume of albuterol, though the amount of time may vary depending on the model of the nebulizer used. Thus, in one day, an individual may be required to spend an hour or more to receive the necessary dosage of albuterol to induce bronchodilation or obtain relief of bronchospasm associated with pediatric asthma, for example. The time requirements for nebulization therapy can be burdensome, and cause individuals to skip required dosages during the day. The impact of not following the prescribed dosage regimen could compromise the individual's condition.

In one alternative embodiment, the volume of the albuterol/ipratropium inhalation solutions of the present invention is about 0.1 ml to about 2.25 ml, or about 0.1 ml to about 2 ml, or about 1 ml to about 2 ml, or about 1.5 ml to about 2 ml, preferably about 0.25 ml or about 0.50 ml. In another alternative embodiment, the volume of the albuterol/ipratropium inhalation solution of the present invention is about 0.05 ml to about 1.0 ml; 0.1 ml to about 0.9 ml; 0.1 ml to about 0.8 ml; 0.1 ml to about 0.7 ml; 0.1 ml to about 0.6 ml; 0.1 ml to about 0.5 ml; 0.1 ml to about 0.4 ml; 0.1 ml to about 0.3 ml; 0.1 ml to about 2.0 ml. In one preferred embodiment the fill volume of the albuterol/ipratropium inhalation solution of the present invention is from about 0.05 ml to about 0.5 ml, preferably from about 0.1 ml to about 0.5 ml, more preferably about 0.25 ml or 0.50 ml. While no clinical trials or other experiments were carried out on these volumes, it is believed that such volumes would be more beneficial over conventional nebulizer solutions (e.g. 2.5 ml or 3.0 ml fill volume) because they will enable the individual to receive more medication (e.g., albuterol) in less time during each nebulization treatment. Also, it is believed that lower fill volumes of the present invention will minimize common handling complications with nebulizer therapy, and it may extend the life of the nebulizer.

In one alternative embodiment, the fill volumes of the present invention may reduce the time of each nebulization treatment by at least 20%, 30%, 40%, 50%, 60%, 70% or 80% or more over conventional nebulizer treatments (e.g. 2.5 ml or 3 ml fill volume). In another alternative embodiment, the fill volumes of the present invention may reduce each nebulization treatment to about or less than about 12, 11, 10, 9, 8, 7, 6, 5, 4, 3, 2, 1 minutes, or any range therebetween less over conventional nebulizer treatments (e.g. 2.5 ml or 3.0 ml fill volume). Reducing the amount of time to complete the treatment means individuals will be more likely to comply with the prescribed dosing regimen and achieve optimal benefit from the medication prescribed.

Another drawback of conventional nebulizer treatments is the loss of medication during administration. Conventional nebulizer solutions comprise about 2.5 ml fill volume of inhalation solution, or more. For example, when nebulizing an inhalation solution using a conventional nebulizer; wherein the solution comprises 2.5 ml or more, about 0.7 ml of the solution remains in the nebulizer system after treatment, though the amount may vary depending on the model of the nebulizer used. In these instances, the individual is not receiving the prescribed dosage or optimum dosage of inhalation medication. For example, in one day, due to the residual medication remaining in the nebulizer system after each treatment, an individual fails to receive approximately 2.1 ml, or more of the prescribed daily amount of medication.

It is believed that the fill volumes of the albuterol/ipratropium inhalation solutions of the present invention will result in lesser amounts of solution remaining in the nebulizer system after treatment, when compared to conventional inhalation solutions (e.g. 2.5 ml or 3 ml fill volume). Less solution remaining in the nebulizer system means more medication (e.g., albuterol and ipratropium) administered to the individual during each treatment. In one alternative embodiment, the amount of solution remaining in the nebulizer system after each treatment may be less than 0.50 ml, or less than 0.30 ml, or less than 0.20 ml or less than 0.10 ml or less than 0.05 ml of the albuterol inhalation solution of the present invention, e.g. an inhalation solution comprising 2.5 mg albuterol.

Important factors to effective nebulizer treatment is deep inspiration to ensure deep penetration of the medication into the lungs, and steady breath-holding to ensure good retention of the medication in the lungs. It is believed that administering a fill volume less than 2.0 ml, preferably from about 0.1 ml to about 0.5 ml, more preferably about 0.25 ml or 0.5 ml of an inhalation solution into a nebulizer, for example, will optimize the therapeutic effect of the individual's deep inspiration efforts during treatment, and will optimize the therapeutic effect of the individual's breath-holding efforts as well. This is due to the shorter treatment time and increased concentration of the albuterol in the solution.

Accordingly, in one alternative embodiment, the present invention is a method of facilitating patient care, reducing medication error, reducing nebulizer treatment time, improving the efficiency and efficacy of nebulizing therapy or enhancing therapeutic compliance of an individual suffering from pediatric asthma. In one alternative embodiment, such method may comprise the step of placing about 0.1 ml to about 2.0 ml of the albuterol inhalation solutions of the present invention into a chamber of a nebulizer. The nebulizer having a mouthpiece or facemask associated with the chamber of the nebulizer. The mouthpiece or facemask is positioned in close proximity to the individual's mouth or face. The inhalation solution may be passed in a mist form from the nebulizer chamber through the mouthpiece or facemask to the individual while the individual breathes into the mouthpiece or facemask. The individual continues breathing into the mouthpiece or facemask until the nebulization treatment is finished. This may take about or less than about 12, 11, 10, 9, 8, 7, 6, 5, 4, 3, 2, 1 or 0.5 minutes, or any range therebetween. In another alternative embodiment, the treatment may be finished in about 60, 50, 40, 30, 20, 10, 5 or 1 second, or any range therebetween. In an alternative embodiment, the nebulization treatment is finished when at least substantially all the mist is removed from the nebulizer chamber. This may take about or less than about 12, 11, 10, 9, 8, 7, 6, 5, 4, 3, 2, 1 or 0.5 minutes, or any range therebetween. In an alternative embodiment, it may take about 60, 50, 40, 30, 20, 10, 5 or 1 second, or any range therebetween.

In another alternative embodiment, the system of the present invention comprises one or more dispensing containers prefilled with about 0.1 ml to about 2.0 ml, or about 0.1 ml to about 1.0 ml; 0.1 ml to about 0.9 ml; 0.1 ml to about 0.8 ml; 0.1 ml to about 0.7 ml; 0.1 ml to about 0.6 ml; 0.1 ml to about 0.5 ml; 0.1 ml to about 0.4 ml; 0.1 ml to about 0.3 ml; 0.1 ml to about 2.0 ml; about 0.5 ml to about 2.0 ml of a premixed, premeasured, aqueous inhalation solution comprising a single unit dose of a therapeutically effective amount of albuterol.

In one preferred embodiment, the present invention comprises 0.05 ml to about 1.0 ml, preferably from about 0.1 ml to about 0.5 ml, more preferably about 0.25 ml or 0.5 ml. The amount of albuterol may range from about 0.60 mg to about 1.25 mg. The solution may be suitable for nebulization in a nebulizer, and the solution may be stable, in that the inhalation solution is therapeutically effective following storage for 12 months at 25° C., for example. Also, in another embodiment, the inhalation solution in each of the one or more containers comprise a preservative or any other suitable anti-microbial agent, such as benzalkonium chloride, or may be preservative free. The inhalation solution may further comprise sodium chloride, water, and an acid to adjust the pH of the inhalation solution to about 4, preferably about 3.5.

TABLE 4

| Type of Asthma | Description |
|---|---|
| Severe Persistent | Continual symptoms during the day; |
| | Frequent symptoms at night; |
| | 60% of lower predicted values for DEV; |
| | and >60% or lower of personal best PEF, |
| | and more than 30% PEF variability. |
| Moderate Persistent | Some symptoms every day; |
| | Nighttime symptoms five or more nights a month; |
| | and >60% and <80% of PEF or FEV₁ and greater |
| | than 30% FEV₁ variability. |
| Mild Persistent | Daytime symptoms 3-6 days a week; |
| | Symptoms at night 3-4 times a month; and |
| | 80% or higher PEF or FEV₁ and PEF variability |
| | of 20-30%. |
| Mild Intermittent | Daytime symptoms 2 or fewer times a week; |
| | Nighttime symptoms 2 or fewer times a month; |
| | and 80% or higher PEF or FEV₁ and PEF |
| | variability <20%. |

In the present invention, a therapeutically effective pediatric amount of albuterol is administered to induce bronchodilation and/or provide relief of bronchospasm in pediatric patients with asthma. Such amount of albuterol may be administered to a pediatric patient after the onset of bronchospasm to reduce breathing difficulties resulting from asthma. In another embodiment, the albuterol may be administered prophylactically, that is, to prevent or to reduce the extent of bronchospasm.

The quantity of albuterol to be administered will be determined on an individual basis, and will be based at least in part on consideration of the patient's size, the severity of the symptoms to be treated and the results sought. The actual dosage (quantity of albuterol administered at a time) and the number of administrations per day will depend on the mode of administration, such as inhaler, nebulizer or oral administration. For example, about 0.63 mg to about 1.25 mg of albuterol given by nebulization one or more times per day would be adequate to produce the desired bronchodilation effect in most children.

In an alternative embodiment, the inhalation solution of the present invention provides relief of bronchospasm in patients 2 to 12 years of age. For example, a 0.63 mg unit dose of albuterol inhalation solution is effective for children ages 10 years and younger, children weighing ≦40 kg or children with less severe asthma. A 1.25 mg unit dose of albuterol is effective for prolonged use in children ages 11-12 years, children weighing >40 kg or in children with more severe asthma.

Further, the albuterol inhalation solution of the present invention may be administered together with one or more other drugs. For example, an antiasthmatic drug such as theophylline or terbutaline, or an antihistamine or analgesic such as aspirin, acetaminophen or ibuprofen, may be administered with or in dose temporal proximity to administration of a therapeutically effective pediatric amount of albuterol. The albuterol and the one or more drugs may be administered in one formulation or as two separate entities. According to the present invention, a therapeutically effective pediatric amount of albuterol, alone or in combination with another drug(s), may be administered to a pediatric individual periodically as necessary to reduce symptoms of asthma.

In an alternative embodiment of the present invention, relief of severe persistent asthma may include administration of a therapeutically effective pediatric amount of albuterol for quick relief of symptoms and a high dose of inhaled corticosteroids using a spacer or holding chamber with a face mask. If needed, oral corticosteroids (2 mg/kg/day) may be administered. Oral corticosteroid should be reduced to the lowest daily or alternate-day dose that stabilizes symptoms.

For moderate persistent asthma, treatment may include administering therapeutically effective pediatric amounts of albuterol for quick relief of symptoms and an inhaled corticosteroid at a mid-level dose, delivered using a spacer or holding chamber with a facemask. As symptom control is achieved, the inhaled corticosteroid dose may be lowered, and either inhaled nedocromil or theophylline may be added.

For mild persistent asthma, treatment may include administering therapeutically effective pediatric amount(s) of the present formulation for quick relief of symptoms and daily anti-inflammatory medication such as low-dose inhaled corticosteroid using a spacer or holding chamber with a facemask or a trial of cromolyn by nebulizer or nedocromily by MDI. For mild intermittent asthma, aside from administering a therapeutically effective pediatric amount of albuterol, no daily drug therapy is ordinarily required.

In another alternative embodiment, the inhalation solution of the present invention may be administered by nebulizer, such nebulizer including, but not limited to, a jet nebulizer, ultrasonic nebulizer and breath-actuated nebulizer. Preferably, the nebulizer is a jet nebulizer connected to an air compressor with adequate air flow. The nebulizer being equipped with a mouthpiece or suitable face mask.

In an alternative embodiment, the system and/or kit of the present invention comprises an inhalation solution comprising a therapeutically effective pediatric amount of albuterol in a prepackaged, premeasured, premixed and/or single unit dose form for the relief of bronchospasm in children. The inhalation solution may be sterile and/or antimicrobial preservative-free.

In another embodiment, the present invention provides a system and/or kit for organizing and storing one or more prefilled dispensing containers, each container comprising a premixed, premeasured inhalation solution comprising a single unit dose of a therapeutically effective pediatric amount of albuterol. Such system and/or kit may provide such containers in prepackaged form. The one or more containers may be comprised of plastic including, but not limited to, a semi-permeable plastic such as, for example, LDPE. The container may also comprise a Twist-Flex™ top, such top comprising an easy-to-grip tab-like handle such that the container may be opened, for example, by twisting off the tab by hand. The Twist-Flex™ top is advantageous in that it allows for easy dispensing of the solution, prevents spillage and eliminates the need to open the container by cutting off the top, or the like, thereby reducing cross-contamination. In one alternative embodiment, the design of the container substantially conforms to those designs illustrated in U.S. Pat. Des. Nos. 317,715; 296,869; 289,609; or 275,732, which are incorporated herein by reference. One or more of the semi-permeable single unit dose containers may be disposed in a sealed aluminum foil pouch, such that the foil provides a protective barrier against environmental contaminants and light. Such a barrier improves the shelf-life and stability of the inhalation solution.

In another alternative embodiment, the present invention comprises a prepackaged inhalation system and/or kit suitable for pediatric patients suffering from asthma. Such prepackaged system and/or kit comprising: (a) one or more single unit dosages of a therapeutically effective pediatric amount of albuterol; (b) administration instructions for the use of said unit dose as an asthma treatment for pediatrics; and (c) a dispensing container prefilled with the one or more single unit doses of albuterol.

In another alternative embodiment, the prepackaged inhalation system and/or kit of the present invention provides one or more premixed, premeasured, single unit dose vials comprising a therapeutically effective pediatric amount of albuterol for the relief of bronchospasm associated with pediatric asthma, and instructions for using the same.

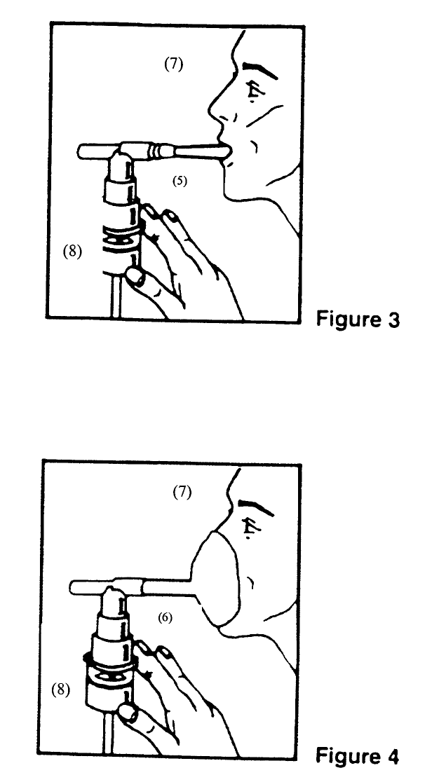

The prepackaged inhalation system and/or kit may be provided in one of any number of forms, including, but not limited to, a box containing one or more prepackaged, unit dose vials or a box containing individual packages or pouches comprising one or more unit dose vials. For example, an embodiment of a unified prepackaged system and/or kit for relieving bronchospasm in children is depicted in FIG. 5. Specifically, FIG. 5 depicts a support package, box, carton or container (10) comprising one or more prepackaged, pre-filled dispensing containers (21-25). Each container comprising a premixed, premeasured inhalation solution. The inhalation solution comprising a unit dose of a therapeutically effective pediatric amount of albuterol for relieving bronchospasm in a child suffering from asthma. The inhalation solution may be provided in sterile and/or antimicrobial preservative-free form.

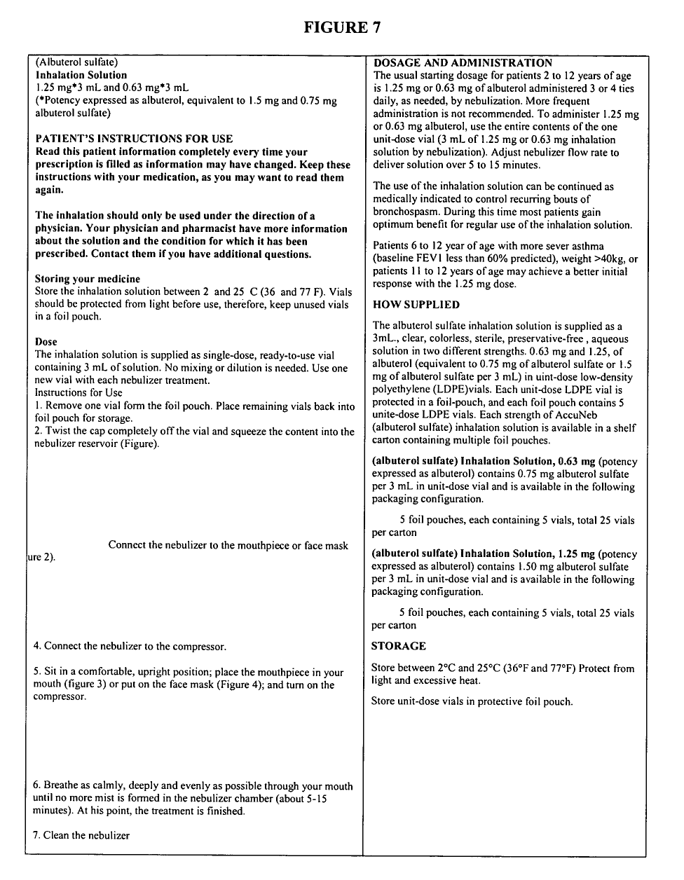

Support package, box, carton or container (10) may incorporate one or more labels (13) therein. One or more labels (13) may comprise indicia (14) indicating that the inhalation solution can be used to relieve bronchospasm in children. The label may also comprise indicia (15) which provides instructions for using the inhalation solution to relieve bronchospasm in children. As used herein “indicia” includes, but is not limited to, wording, pictures, drawings, symbols and/or shapes. A non-limiting example of the indicia that may appear on the one or more labels (13) is shown in FIG. 7. The one or more labels may be positioned on one or more surfaces of the support package, box, carton or container (10) or a separate sheet, or any combination thereof. Support package (10) may also incorporate lid (16) to enclose the packaging material therein.

The system and/or kit of the present invention may also include a label and/or instructions designed to facilitate user compliance. For example, in an embodiment, a system and/or kit of the present invention comprises packaging material containing one or more prepackaged vials comprising a sterile, premixed, premeasured, unit dose of an inhalation solution comprising a therapeutic effective pediatric amount of albuterol. The packaging material may further comprise a label indicating that each vial can be used with each nebulizer treatment for the relief of bronchospasm associated with pediatric asthma. Such instructions may also include instructions on dosage for each nebulizer treatment, as well as instructions for administration, such as by nebulizer. The instructions may be positioned on one or more surfaces of the packaging material, or the instructions may be provided on a separate sheet, or any combination thereof.

In an alternative embodiment, the present invention is directed to a prepackaged therapeutic system and/a kit for inducing bronchodilation in a child suffering from asthma, the prepackaged therapeutic system comprising:
(a) one or more dispensing containers; the one or more containers each prefilled with about 3 ml of a sterile, benzalkonium chloride-free, premixed, premeasured aqueous inhalation solution comprising a unit dose of a therapeutically effective pediatric amount of racemic albuterol; wherein the dosage of racemic albuterol is about 0.63 or about 1.25 mg; the inhalation solution in each of the one or more containers is suitable for nebulization in a nebulizer; wherein the inhalation solution in each of the one or more containers has a long shelf life;
(b) indicia comprising indication, adverse reaction, dosage and administration data pertaining to the inhalation solution in each of the one or more containers;
(c) wherein the indication data comprises data that the inhalation solution in each of the one or more containers is indicated for the relief of bronchospasm in patients 2 to 12 years of age with asthma; and
(d) wherein the adverse reaction data comprises data indicating that otitis media and skin-appendage infection might occur after administering the inhalation solution in the one or more containers.

In another alternative embodiment, the prepackaged therapeutic system of the present invention comprises data that the dosage for patients 2 to 12 years of age is 0.63 mg or 1.25 mg of albuterol administered 3 to 4 times daily by nebulization over 5 to 15 minutes. Also, the adverse reaction data may include a list of one or more preprinted adverse events that may occur after administering the inhalation solution in each of the one or more containers, the adverse events comprising asthma exacerbation, allergic reaction, gastroenteritis, flu syndrome, lymphadenopathy, urticaria, migraine, chest pain, bronchitis or nausea.

In another alternative embodiment, the prepackaged therapeutic system and/a kit is adapted to induce bronchodilation in a child suffering from asthma, the prepackaged therapeutic system may comprise:
(a) one or more dispensing containers; the one or more containers each prefilled with about 3 ml of a sterile, stable, benzalkonium chloride-free, premixed, premeasured aqueous inhalation solution consisting essentially of a unit dose of a therapeutically effective pediatric amount of racemic albuterol; wherein the dosage of racemic albuterol is about 0.63 or about 1.25 mg; wherein the racemic albuterol is in the form of an acid addition salt; wherein the acid addition salt is albuterol sulfate; the inhalation solution in each of the one or more containers is suitable for nebulization in a nebulizer; wherein the inhalation solution in each of the one or more containers has a long shelf life;
(b) indicia comprising indication, adverse reaction, and dosage and administration data pertaining to the inhalation solution in each of the one or more containers;
(c) wherein the indication data comprises data that the inhalation solution in each of the one or more containers is indicated for the relief of bronchospasm in patients 2 to 12 years of age with asthma;
(d) wherein the adverse reaction data comprises a list of preprinted adverse events that may occur after administering the inhalation solution in each of the one or more containers; the adverse events comprising otitis media, skin-appendage infection, asthma exacerbation, allergic reaction, gastroenteritis, flu syndrome, lymphadenopathy, urticaria, migraine, chest pain, bronchitis or nausea; and
(e) wherein the dosage and administration data comprises data that the dosage for patients 2 to 12 years of age is 0.63 mg or 1.25 mg of albuterol administered 3 to 4 times daily by nebulization over 5 to 15 minutes.

The present invention is also directed to a method of treating bronchospasm associated with pediatric asthma, wherein albuterol is administered as a unit dose from about 0.63 mg to about 1.25 mg of albuterol. Such unit dose may be in the form of a nebulizer solution.

In an alternative embodiment, the method of the present invention comprises the step of administering to a patient 2 to 12 years old in need thereof an inhalation solution comprising a therapeutically effective pediatric amount of albuterol. Such solution may comprise from about 0.63 mg to about 1.25 mg albuterol. Such solution may also be premixed, premeasured, antimicrobial preservative-free and/or sterile. Such solution may also be in a single unit dose vial.

In another alternative embodiment, the method of the present invention comprises the step of administering to a pediatric patient in need thereof an inhalation solution comprising a therapeutically effective pediatric amount of albuterol. The inhalation solution being administered by nebulizer, more preferably a jet nebulizer connected to an air compressor with adequate air flow.

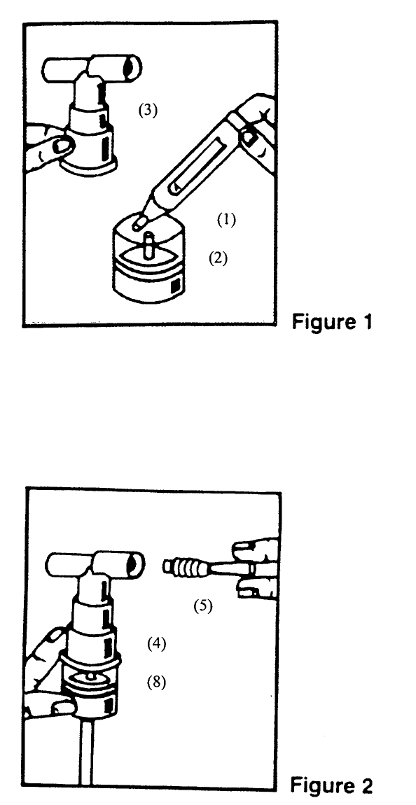

In yet another alternative embodiment, in reference to FIGS. 1-4, the method of the present invention comprises the steps: (i) placing an inhalation solution comprising a therapeutically effective pediatric amount of albuterol (1) into a nebulizer cup (2) the nebulizer may be powered by attachment to compressed gas cylinders or an electrically driven compressor; (ii) using a “T” adapter (3) to fit the cup lid (4) to a mouthpiece (5) or facemask (6); (iii) drawing the albuterol solution up by the velocity of a gas jet and fragmenting it into an aerosol; (iv) passing the aerosol through the mouthpiece (5) or facemask (6) to the pediatric patient (7) afflicted with bronchospasm; and (v) the patient continues breathing until no more mist is formed in the nebulizer chamber (8). This may occur in about 5-15 minutes.

In one alternative embodiment, the usual starting dosage for patients 2 to 12 years of age is about 1.25 mg or about 0.63 mg of albuterol administered 3 or 4 times daily, as needed by nebulization. To administer these amounts of albuterol, the entire contents of a one unit-dose vial (e.g., 1.50 mg/3 ml or 0.75 mg/3 ml albuterol sulfate) may be used by nebulization. Preferably, the nebulizer flow rate is adjusted to deliver the albuterol sulfate over 5 to 15 minutes. Patients 6 to 12 years of age with more severe asthma (baseline FEV₁ less than 60% predicated), weight >40 kg or patients 11 to 12 years of age may achieve a better initial response with about a 1.25 mg dose.

Further, in an alternative embodiment, the method of the present invention comprises the steps: (i) preparing an inhalation solution comprising a therapeutically effective pediatric amount of albuterol solution by diluting one or more solutions comprising albuterol; and (ii) administering the inhalation solution to a pediatric patient in need thereof.

In another alternative embodiment, the present invention is directed to a method of inducing bronchodilation in a child suffering from asthma, said method comprising the step of:
(a) providing the child or prescriber a prepackaged therapeutic system comprising:
   one or more dispensing containers; the one or more containers each prefilled with about 3 ml of a sterile, stable, benzalkonium chloride-free, premixed, premeasured aqueous inhalation solution comprising a unit dose of a therapeutically effective pediatric amount of racemic albuterol; wherein the dosage of racemic albuterol is about 0.63 or about 1.25 mg; the inhalation solution in each of the one or more containers is suitable for nebulization in a nebulizer; wherein the inhalation solution in each of the one or more containers has a long shelf life;
(b) providing the child or prescriber of the prepackaged therapeutic system indication, adverse reaction, dosage and administration data pertaining to the inhalation solution in each of the one or more containers;
(c) wherein the indication data informs the patient or prescriber that the inhalation solution in each of the one or more containers is indicated for the relief of bronchospasm in patients 2 to 12 years of age with asthma; and
(d) wherein the adverse reaction data informs the patient or prescriber that otitis media and skin-appendage infection might occur after administering the inhalation solution in the one or more containers.

In another alternative embodiment, the dosage and administration data informs the patient or prescriber that the dosage for children 2 to 12 years of age is 0.63 mg or 1.25 mg of albuterol administered 3 to 4 times daily by nebulization over 5 to 15 minutes. Also, in the adverse reaction data may include a list of one or more preprinted adverse events that may occur after administering the inhalation solution in each of the one or more containers, the adverse events comprising asthma exacerbation, allergic reaction, gastroenteritis, flu syndrome, lymphadenopathy, urticaria, migraine, chest pain, bronchitis or nausea.

In another embodiment, the method of the present invention is directed to inducing bronchodilation in a child suffering from asthma. Such method may comprise the step of:
(a) providing the child or prescriber a prepackaged therapeutic system comprising:
   one or more dispensing containers; the one or more containers each prefilled with about 3 ml of a sterile, stable, benzalkonium chloride-free, premixed, premeasured aqueous inhalation solution consisting essentially of a unit dose of a therapeutically effective pediatric amount of racemic albuterol; wherein the dosage of racemic albuterol is about 0.63 or about 1.25 mg; wherein the racemic albuterol is in the form of an acid addition salt; wherein the acid addition salt is albuterol sulfate; the inhalation solution in each of the one or more containers is suitable for nebulization in a nebulizer; wherein the inhalation solution in each of the one or more containers has a long shelf life;
(b) providing the child or prescriber of the prepackaged therapeutic system indication, adverse reaction, dosage and administration data pertaining to the inhalation solution in each of the one or more containers;
(c) wherein the indication data informs the patient or prescriber that the inhalation solution in each of the one or more containers is indicated for the relief of bronchospasm in patients 2 to 12 years of age with asthma;
(d) wherein the adverse reaction data informs the patient or prescriber that otitis media, skin-appendage infection, asthma exacerbation, allergic reaction, gastroenteritis, flu syndrome, lymphadenopathy, urticaria, migraine, chest pain, bronchitis or nausea might occur after administering the inhalation solution in the one or more containers; and
(e) wherein the dosage and administration data comprises data that informs the patient or prescriber that the dosage for patients 2 to 12 years of age is 0.63 mg or 1.25 mg of albuterol administered 3 to 4 times daily by nebulization over 5 to 15 minutes.

The present invention also provides a process for making a sterile, premixed, premeasured, and/or BAC-free inhalation solution comprising a single unit dose of a therapeutically effective pediatric amount of albuterol. In such an embodiment, the method of the present invention comprises one or more of the following steps: (i) adding at least a therapeutically effective pediatric amount of albuterol in a vehicle, such as water; (ii) optionally sterilizing the solution and sealing the container. An osmotic adjusting agent may be added to adjust the isotonicity of the solution. In one embodiment of the present invention, the solution of the present invention is isotonic. Isotonicity may be achieved by adding an osmotic adjusting agent to adjust the isotonicity of the solution from about 280 to about 320 mOsm/kg. In addition, an acid (e.g., sulfuric acid) may be added to adjust the pH of the solution to a level ranging from about 3.0 to about 4.0, preferably about 3.5. The method of the present invention may further comprise the step of adding hydrochloric acid to adjust the pH of the inhalation solution.

In another embodiment, a process for making an inhalation solution of the present invention comprises one or more of the following steps: (i) adding at least a therapeutically effective pediatric amount of albuterol in a vehicle such as water; (ii) placing the mixture in a container, and sterilizing the mixture by steam sterilization, or any other sterilizing means known in the art. Each mixture being filled into a vial, and then packaged, stored and/or used directly. Here, the resulting mixture is stable, and after sterilization, it can be dispersed, if necessary, into multiple mixtures each containing a unit dose of a therapeutically effective pediatric amount of albuterol.

Osmotic adjusting agents which may be used include, but are not limited to, sodium chloride, potassium chloride, zinc chloride, calcium chloride and mixtures thereof. Other osmotic adjusting agents may also include, but are not limited to, mannitol, glycerol, and dextrose and mixture thereof. In an alternative embodiment, the present invention may comprise about 0.4 to about 1.0 weight percent ionic salt. Preferably, the present invention comprises about 0.9 weight percent of an osmotic adjusting agent.

The present method may further require filling the solution into one or more dispensing vials, each vial being filled with about 0.1 ml to about 5 ml, or about 0.1 ml to about 2.25 ml, or about 0.1 ml to about 3.0 ml, about 0.5 ml to about 3.0 ml, or about 0.5 ml to about 2.0 ml, or about 0.1 ml to about 2 ml, preferably about 1 ml, about 2 ml, or about 3 ml of the solution such that the solution in the each vial comprises a unit dose of a therapeutically effective amount of albuterol and ipratropium bromide. Also, in another alternative embodiment, the stability of the solution in the one or more dispensing containers is such that the solution is therapeutically effective following storage for 12 months at 25° C. The solution may be suitable for nebulization in a nebulizer.

In an alternative embodiment, the inhalation solution of the present invention may be prepared as follows: (i) fitting a high density polyethylene (HDPE) or stainless steel formulation tank with a bottom drain and peristaltic recirculation system (for HDPE) or tri-blender (for stainless steel) for mixing; (ii) filling the tank with approximately 90% of the required amount of Purified Water USP at a temperature of between 18° C. to 25° C.; while mixing, (iii) adding sulfuric acid, Sodium Chloride USP, and at least a therapeutically effective pediatric amount of Albuterol Sulfate USP to the tank; (iv) continue mixing until all chemical components are dissolved; (v) adding Purified Water USP to adjust the final volume, if necessary, thus producing an albuterol mixture.

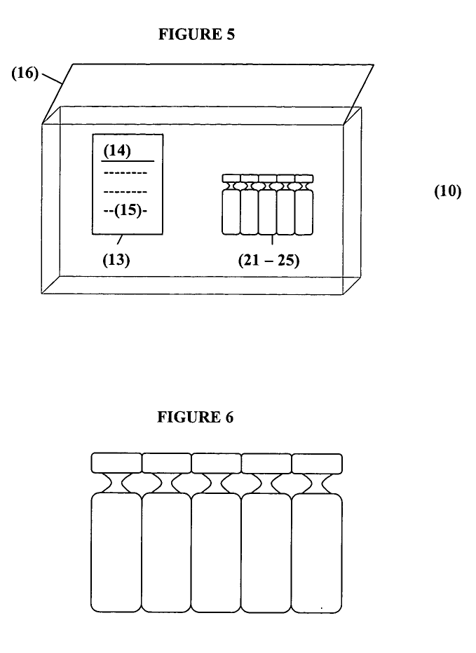

From the formulation tank, the albuterol mixture is pumped through sanitary delivery lines directly into a form-fill-seal (FFS) machine. The albuterol mixture passes through a 0.2 micron sterilizing cartridge filter, to the filling nozzles within the sterile air shower compartment, and subsequently into formed vials of low density polyethylene (LDPE). The albuterol mixture being sterile filled into the vials such that each vial contains a single unit dose of a therapeutically effective amount of albuterol. The filled vials are then sealed. The machine may form, fill and seal the vials in a continuous operation under aseptic conditions, thus producing a sterile product. For example, cards of five filled vials (FIG. 6) are overwrapped into a protective laminated foil pouch using an autowrapper machine. Five or twelve such pouches may then be packaged in a shelf carton, thus forming a prepackaged therapeutic system for relieving bronchospasm in children suffering from asthma. An appropriate label and instructions may be added in the shelf carton.

The present invention is also directed to a method of forming a unit-dose nebulizer solution comprising the step of: (i) preparing an admixture containing a therapeutically effective pediatric amount of albuterol in a pharmaceutically acceptable vehicle.

In an alternative embodiment, the present invention also comprises a device for use in the relief of bronchospasm associated with pediatric asthma. Such device may take the form of a label, written instructions or any other form incorporating indicia thereon. The device may comprise indicia which indicates that a patient suffering from bronchospasm can be treated with at least one prepackaged, sterile, premixed, premeasured and/or antimicrobial preservative-free inhalation solution comprising a unit dose of a therapeutically effective pediatric amount of albuterol in a single vial. The inhalation solution being suitable for nebulization in a nebulizer. The device also comprising indicia which provides instructions for utilizing the inhalation solution to relieve said bronchospasm in the patient.

### VII. EXAMPLES

### Example 1

Patients were randomized to receive a nebulizer solution comprising either 0.63 mg/3 ml or 1.25 mg/3 ml of albuterol sulfate, or a placebo. The inhalation solution was administered via a Pari LC Plus™ nebulizer and a Pari PRONEB™ compressor. Both of these products are commercially available.

In this study, qualifying children ages 6 to 12 were randomized to receive 1 of the following three treatments twice daily (TD) for 4 weeks, each in 3.0 mL volume: (1) 1.25 mg albuterol sulfate inhalation solution; (2) 9.63 mg albuterol sulfate inhalation solution; or (3) placebo (saline). Each patient was provided with a personal compressor-driven PARI LC PLUS™ nebulizer, by Pari Respirator Equipment, Inc., Richmond, Va., for the duration of the study.

A screening visit was followed by a 2-week placebo run-in phase to confirm the need for regular symptomatic beta-agonist therapy, and to give patients experience with daily diaries and peak flow measurements, as well as to demonstrate compliance. The 4-week study period began with the initial dose, to be taken in the morning, administered at the study site. Pre-dose pulmonary function tests and pulmonary function tests 30 minutes after the end of nebulization and hourly thereafter for 6 hours were performed.

After 11 days, patients returned for exchange of study medication and diaries and pulmonary function tests before and 30 minutes after the morning dosing. After completing 28 days of treatment, patients returned to the test site for a repeat of the 6-hour evaluation of safety and efficacy following administration of study medication. Diary cards were used to record asthma symptoms, night awakenings, peak flow measurements, supplemental albuterol use, change in medication and adverse events. The safety profiles of each unit dose and placebo were determined by collecting vital signs (heart rate, blood pressure, respiration rate, and body temperature) as well as electrocardiograms.

Patients

A total of 349 children (220 males and 129 females) were initially randomized, and 288 completed the double-blind 4-week treatment period. Demographic and other baseline characteristics were comparable between the three treatment groups. To be eligible for enrollment, patients had to meet the criteria described in Table 3 below.

TABLE 5
Inclusion/Exclusion Criteria

| Design Element | Description |
|---|---|
| Inclusion Criteria | Documented history (26 months) of moderately severe |
| | persistent asthma confirmed by a physician and |
| | requiring daily asthma medication. |
| | Generally good health apart from asthma. |
| | FEV₁ between 50% and 80% of predicted values at |
| | baseline and at the beginning of the double-blind |
| | treatment phase. |
| | At least 15% reversibility in FEV₁ following the |
| | administration of inhaled nebulized albuterol |
| | at the screening visit. |
| | Symptomatic asthma requiring the use of beta- |
| | agonists on at least 6 of the 14 days of |
| | observation during the placebo-controlled |
| | run-in-period. |
| | Willingness of patient and caregiver to provide |
| | informed consent. |
| Exclusion Criteria | Severe asthma or any serious medical condition. |
| | Use of prescription medication for which albuterol |
| | sulfate is contraindicated. |
| | Known hypersensitivity to albuterol of similar |
| | agents. |
| | Active pulmonary disease other than bronchial |
| | asthma. |
| | Upper respiratory tract infection within 4 weeks |
| | of the start of the placebo phase. |
| | Any other chronic condition that could have |
| | interfered with successful completion of |
| | the study or confounded its interpretation. |
| | Acute use of corticosteroids or other treatments |
| | which might interfere with the study within 4 |
| | weeks of the screening visit. |
| | Inability or unwillingness to perform the require- |
| | ments of the protocol. |

Interventions

Patients meeting the inclusion criteria and on regularly prescribed asthma medications were permitted to continue on those medications during the course of the study if the doses remained stable. Patients were required to withhold their morning dose before each study visit and during the entire study session. After the patient completed the study session, the regularly scheduled dosing resumed for that day. All medication used to treat chronic conditions, including immunotherapy, had to be initiated at least 30 days prior to the start of the study, and the dosing regimen had to be stabilized by the initial visit. Racemic albuterol delivered by a chlorofluorocarbon (CFC) MDI or nebulizer was used on an as-needed basis as the rescue medication.

Efficacy Results

The primary efficacy endpoint was the area under the percent change from pre-dose FEV, versus time curve for the initial closing visit (Day 1) and the final closing visit (Day 28). Compared to placebo, both unit doses of albuterol produced significant improvement in FEV, following both the initial dose and the dose given at visit 4 after 4 weeks of TD treatment. The mean percent change from baseline in the area under the 6-hour curve for FEV₁ for both active treatment regimens compared with placebo, is shown in Table 4 (for Day 1) and Table 5 (for Day 28).

[image]

[image]

The onset of a 15% increase in FEV₁ over baseline for both doses of AccuNeb was seen at 30 minutes. The mean time to speak effect was approximately 30 to 60 minutes for both doses on day 1 and after 4 weeks of treatment. The mean duration of effect, as measured by a >15% increase from baseline in FEV₁ was approximately 2.5 hours for both doses on day 1 and approximately 2 hours for both doses after 4 weeks of treatment. In some patients, the duration of effect was as long as 6 hours.

Subgroup analysis was performed to determine whether the overall efficacy of AccuNeb was consistent across all age, weight and disease severity groups. In all age groups, weight categories and disease severity groups, the 1.25 mg dose provided a statistically significant improvement over placebo on both Day 1 and Day 28. However, at the lower 0.63 mg dose, children 11 to 12 years of age, children heavier than 40 kg and children with more severe disease (classified as an FEV₁ ≦60% of predicted) did not have a statistically significant improvement in FEV₁ over placebo at Day 29. As a result, older children, heavier children or children with more severe disease may have a better response at the 1.25 mg dose.

Safety/Tolerability

Adverse reaction information to the albuterol solution used in the study was derived from the 4-week controlled clinical trial described above. Adverse events were reported in >1% of patients receiving the present solution, more frequently than adverse events reported by patients receiving placebo, as shown in Table 6. In the study, there was one case of ST segment depression in the 1.25 mg treatment group, but no clinically relevant laboratory abnormalities related to administration were observed.

TABLE 8
Adverse Event Reports
(ADVERSE EVENTS WITH AN INCIDENCE OF 0.1%
OF PATIENTS RECEIVING THE PRESENT ALBUTEROL
SOLUTION AND GREATER THAN PLACEBO
(EXPRESSED AS % OF TREATMENT GROUP))

| | 1.25 mg | 0.63 mg | |
|---|---|---|---|
| | AccuNeb | AccuNeb | Placebo |
| | (N = 115) | (N = 117) | (N = 117) |
| Asthma Exacerbation | 13 | 11.1 | 8.5 |
| Otitis Media | 4.3 | 0.9 | 0 |
| Allergic Reaction | 0.9 | 3.4 | 1.7 |
| Gastroenteritis | 0.9 | 3.4 | 0.9 |
| Cold Symptoma | 0 | 3.4 | 1.7 |
| Fly Syndrome | 2.6 | 2.6 | 1.7 |
| Lymphadenopathy | 2.6 | 0.9 | 1.7 |
| Skin/Appendage | 1.7 | 0 | 0 |
| injection | | | |
| Urticaria | 1.7 | 0.9 | 0 |
| Migrane | 0.9 | 1.7 | 0 |
| Chest Pain | 0.9 | 1.7 | 0 |
| Bronchitis | 0.9 | 1.7 | 0.9 |
| Nausea | 1.7 | 0.9 | 0.9 |

The figures and attachments herein are presented for illustrative proposes only. They are not intended to limit the scope of the invention. Further, it should be understood that various changes and modifications to the presently preferred embodiment described herein will be apparent to those skilled in the art. Such changes and modifications can be made without departing from the spirit and scope of the present invention and without diminishing its attendant advantages. It is therefore intended that such changes and modifications be covered by the appended claims.

Also, the invention may suitably comprise, consist of or consist essentially of the elements described herein. Further, the invention described herein suitably may be practiced in the absence of any element which is not specifically disclosed herein.

### Example 2

Example 2 is a prophetic example of a nebulizable inhalation solution of the present invention having about 0.5 ml fill volume. It is provided to illustrate, but not limit, the present invention. It is believed that prophetic Example 2 would be suitable for inducing bronchodialation or providing relief of bronchospasm in an individual 2 to 12 years suffering from obstructive airway disease such as pediatric asthma. The inhalation solution may be a sterile, premixed, premeasured single unit dose for asthmatic patients 2 to 12 years of age. It may also comprise all other attributes, features and ingredients of the various embodiments of the present invention, as described herein. Prophetic Example 2 may be administered to an individual in accordance with one or more of the modes of administration described herein.

TABLE 9

| Ingredient | Composition (% w/w) | Range (% w/w) |
|---|---|---|
| Albuterol sulfate | About 0.30 or about | 0.1 to 2.5 |
| | 0.15 (expressed | |
| | as sulfate) | |
| EDTA | 0.01 | 0.001 to 0.2 |
| Sodium Chloride | 0.82 | 0 to 0.9 |
| 1N HCl | 0.046 | 0 to 1.4 |
| Purified water | q.s. | q.s. |

1. A system for providing relief of bronchospasm in an individual 2 to 12 years suffering from obstructive airway disease, said system comprising:
(a) at least one single dispensing container; said container prefilled with about 0.1 ml to about 1.0 ml of a premixed, premeasured, aqueous inhalation solution comprising a single unit dose of a therapeutically effective amount of albuterol; wherein said amount of albuterol ranges from about 0.63 mg to about 1.5 mg; the solution being suitable for nebulization.

2. The system of claim 1, wherein the container is prefilled with about 0.05 ml to about 0.8 ml of the inhalation solution.

3. The system of claim 1, wherein one container is prefilled with about 0.1 ml to about 0.5 ml of the inhalation solution.

4. The system of claim 1, wherein the container is prefilled with about 0.25 ml or about 0.5 ml of the inhalation solution.

5. The system of claim 1, wherein the amount of albuterol is about 0.63 mg or about 1.25 mg.

6. The system of claim 1, wherein the container is prefixed with about 0.5 mml of the inhalation solution, and the amount of albuterol is about 0.63 mg or 1.25 mg.

7. The system of claim 1, wherein the inhalation solution in each of the one or more containers is sterile.

8. The system of claim 1, wherein the inhalation solution in each of the one or more containers is free of benzalkonium chloride.

9. The system of claim 1, wherein the system further comprises a label which indicates that the inhalation solution can be used to relieve broncospasm in individuals 2 to 12 years of obstructive airway disease.

10. The system of claim 9, wherein said label comprises instructions for using the solution to relieve said bronchospasm.

11. The system of claim 9, wherein said label providing prescribing information comprising efficacy, dosage, administration, contraindication and adverse reaction information pertaining to the inhalation solution in the container.

12. A system for reducing medication error, reducing nebulizer treatment time and enhancing therapeutic compliance of an individual 2 to 12 years of obstructive airway disease, the prepackaged therapeutic system comprising:
(a) one or more dispensing containers; the one or more containers each prefilled with about 0.1 ml to about 1.0 ml of a sterile, benzlakonium chloride-free, premixed, premeasured aqueous inhalation solution comprising a unit dose of a therapeutically effective amount of albuterol; wherein the amount of albuterol is about 0.63 mg or 1.25 mg; the inhalation solution in each of the one ore more containers is suitable for nebulization in a nebulizer; the inhalation solution in each of the one or more containers is stable.

13. A method of inducing bronchodialation or providing relief of bronchospasm in an individual 2 to 12 years suffering from obstructive airway disease, said method comprising the step of:
(a) administrating to the individual at least one single dispensing containers; the one or more containers each prefilled with about 0.1 ml to about 1.0 ml of a premixed, premeasured aqueous inhalation solution comprising a unit dose of a therapeutically effective amount of albuterol; wherein the amount of albuterol is about 0.6 mg to about 1.5 mg; the inhalation solution in each of the one or more containers is suitable for nebulization in a nebulizer.

14. The method of claim 13, wherein each of the one ore more containers is prefilled with about 0.1 ml to about 0.8 ml of the inhalation solution.

15. The method of claim 13, wherein each of the one or more containers is prefilled with about 0.1 ml to about 0.5 ml of the inhalation solution.

16. The method of claim 13, wherein each of the one or more containers is prefilled with about 0.25 ml or 0.5 ml of the inhalation solution.

17. The method of claim 13, further comprising the step of providing dosage, administration, contraindication and adverse reaction data pertaining to the inhalation solution in each of the one ore more containers.

18. The method of claim 13, wherein the albuterol is albuterol base.

19. A method of inducing bronchodialation or providing relief of bronchospasm, reducing medication error, reducing nebulization treatment time and enhancing therapeutic compliance of an individual 2 to 12 years suffering from obstructive airway disease, said method comprising the steps:
(a) placing the inhalation solution of claim 1 into a chamber of a nebulizer, said nebulizer having a mouthpiece or facemask associated with the chamber of the nebulizer;
(b) positioning the mouthpiece or facemask in close proximity to the individual's mouth or face;
(c) passing the inhalation solution in a mist form from the nebulizer chamber through the mouthpiece or facemask to the individual while the individual breathes into the mouthpiece or facemask; and
(d) the individual breathing into the mouthpiece or facemask until the nebulization treatment is finished.

20. The method of claim 19, wherein the nebulization treatment is finished when at least substantially all the mist is removed from the nebulizer chamber.

21. The method of claim 19, wherein at least substantially all the mist is removed from the nebulizer chamber in less than 12 minutes.

22. The method of claim 19, wherein at substantially all the mist is removed from the nebulizer chamber in less than 10 minutes.

23. The method of claim 19, wherein at least substantially all the mist is removed from the nebulizer chamber in less than 8 minutes.

24. The method of claim 19, wherein at least substantially all the mist is removed from the nebulizer chamber in less than 6 minutes.

25. The method of claim 19, wherein at least substantially all the mist is removed from the nebulizer chamber in less than 4 minutes.

26. A method of reducing medication error and enhancing therapeutic compliance in an individual 2 to 12 years suffering from obstructive airway disease, said method comprising the steps of:
(a) administering to the individual at least one single dispensing container wherein the container is prefilled with about 0.1 ml to about 0.5 ml of a sterile, benzalkonium chloride-free, premixed, premeasured aqueous inhalation solution comprising a unit dose of a therapeutically effective amount of albuterol; wherein the amount of albuterol ranges from about 0.60 to 1.30 mg; the inhalation solution in the container is suitable for nebulization in a nebulizer; the inhalation solution in the container is stable, in that the inhalation solution is therapeutically effective following storage for 12 months at 25° C.; and
(b) providing dosage information pertaining to the inhalation solution in the container; and
(c) providing administration, information, wherein said administration information comprises instructions for the use of the unit dose of albuterol to treat pediatric asthma.

27. The method of claim 26, wherein the dispensing container is prefilled with 0.25 ml or 0.5 ml.
